(19) United States
(12) Patent Application Publication
(10) Pub. No.: US 2007/0270612 A1
(43) Pub. Date: Nov. 22, 2007

(54) Biocidal azole emulsion concentrates having high active ingredient content

(76) Inventors: Michael P. Pompeo, Sumter, SC (US); Robert L. Hodge, Sumter, SC (US)

Correspondence Address: HAYDEN STONE, PLLC, 101 NORTH COLUMBUS ST., SUITE 409, ALEXANDRIA, VA, 22301, US

(21) Appl. No.: 11/703,818

(22) Filed: Feb. 8, 2007

Related U.S. Application Data
(60) Provisional application No. 60/771,017, filed on Feb. 8, 2006.
(60) Provisional application No. 60/774,655, filed on Feb. 21, 2006.

(51) Int. Cl. C07C 233/00

(52) U.S. Cl. 564/123

(57) ABSTRACT

A concentrated stable emulsifiable formulation includes A) an amide, B) a water-soluble non-ionic emulsifier, C) a water-insoluble anionic emulsifier, and D) a water-insoluble biocidal azole, wherein the concentrated stable emulsifiable formulation is a solution containing the water-insoluble biocidal azole in an amount greater than 20% by weight of the formulation. An aqueous wood treatment composition which can be made for example from the concentrated stable emulsifiable formulation includes emulsified droplets containing at least one water-insoluble biocidal azole s and an amide such as N,N-dialkyloctanamide, N,N-dialkyldecanamide, N-alkyloctanamide, N-alkyldecanamide, octanamide, decanamide, or mixture thereof, where the alkyl groups independently comprise between one and three carbon atoms.

### CROSS-REFERENCE TO RELATED APPLICATIONS:

This application claims priority to provisional applications 60/771,017 filed on Feb. 8, 2006, and 60/774,655 filed on Feb. 21, 2006, the entire disclosures of which are incorporated herein by reference thereto for all legal purposes.

### STATEMENT REGARDING FEDERALLY SPONSORED RESEARCH OR DEVELOPMENT

N/A.

### INCORPORATION-BY-REFERENCE OF MATERIAL SUBMITTED ON A COMPACT DISC: N/A. SEQUENCE LISTING

N/A.

### FIELD OF THE INVENTION

The present invention relates to stable concentrates of organic biocides, in particular biocidal azoles such as tebuconazole, that can be directly added to water to form dispersible emulsions for use in crop, agriculture, and wood preservation, where the concentrate has a high content of active ingredients.

### BACKGROUND OF THE INVENTION

Azoles are registered as pesticides for the use in wood preservation industry, and also used in the agricultural applications to protect plants, fruits, vegetables, and cereal crops from fungal attack. The word wood is meant to be inclusive, that is, to include lumber, timber, posts, wood coverings, veneers, wicker, millwork, joinery, wood products such as plywood, fiberboard, chipboard, waferboard, particleboard, and other wood-containing products used in construction. Freshly milled timber and wood for millwork/joinery are usually treated with preservatives by dipping and/or double vacuum treatments. Wood for use in ground and many above ground applications are usually treated with a preservative by pressure treatment to achieve greater penetration of the preservatives into the wood. Preservatives can also be applied by brushing, spraying, soaking, and similar treatment methods.

Tebuconazole, and similar azoles, are preferred organic biocides for use in wood and also for selected foliar applications and for incorporation into biocidal materials. Exemplary other preferred biocides from the same class of material include propiconazole, cyproconazole, tetraconazole, difenoconazole, diniconazole, penconazole, imibenconazole, propiconazole, azaconazole, and epoxyconazole. Other related biocides include etridiazole, flusilazole, and TCMBT (2-(thiocyanomethylthio)benzothiazole. While there are a number of patents and products that combine a variety of biocidal azoles with copper, a well-known commercial method of preserving wood with copper and an azole known as Wolman E™ (Arch Chemicals Inc.) uses a water soluble formulation called copper azole.

For an organic biocide to be useful in wood, the organic biocide must first be placed into the wood. Generally, the wood preservation industry requires that combinations of various biocides which form a wood preservative package be injected in a single step. For a copper/azole combination to be useful in wood, the azole (e.g., tebuconazole) must 1) be solubilized in water or be dispersed as an injectable micro-emulsion or micro-dispersion in water, 2) be admixed with a source of copper ions, and 3) be injected into wood. Tebuconazole, and similar azoles, are utilized as an emulsion by 1) dissolving the tebuconazole in an oil, solvent, and /or surfactant, and 2) emulsifying the dissolved phase in water by a process where the dissolved tebuconazole is added to the water or a aqueous copper-containing solution such as a copper-amine solution and particularly to a copper/monoethanolamine/carbonate solution with sufficient shear forces, and the resultant tebuconazole emulsion can then be injected into the wood or other material, or sprayed as a solution over crops, foliage, or seeds. Tebuconazole can also be solubilized with large amounts of surfactant.

U.S. Pat. No. 5,468,747 describes a fungicidal composition for use on foliage or seeds comprising two active ingredients, one of which may be tebuconazole, and states that suitable non-ionic agents are the condensation products of ethylene oxide with fatty alcohols such as oleyl or cetyl alcohol, or with alkyl phenols such as octyl- or nonylphenol and octylcresol. Other useful non-ionic agents include partial esters derived from long chain fatty acids and hexitol anhydrides, the condensation products of the said partial esters with ethylene oxide, and lecithins.

U.S. Pat. Nos. 5,527,384 and 5,634,967 describe synergistic interactions between certain biocidal azoles, including tebuconazole, and soluble copper in the field of wood preservation. These patents mention a biocidally active quaternary ammonium compound or tertiary amine salt can be included to aid in the formation of emulsions of biocidal azole compounds in aqueous solutions of the biocidal metal ion, and that the presence of these compounds may mean that additional organic solvents are not necessary to solubilize the biocidal azole compound. The concentrate examples for use with copper and an amine in this patent include a first concentrate having 0.24 parts tebuconazole, 7.76 parts of solvent and oil, and 1 part of an undisclosed anionic/non-ionic emulsifier; a second concentrate having 10% tebuconazole, 50% of ester glycol, 10% of 2-ethyl hexanoic acid, 10% of process oil, and 20% of an undisclosed anionic/non-ionic emulsifier; a third concentrate having 4% benzalkonium chloride and 0.8% tebuconazole; and a fourth concentrate having 4% didecyldimethylammonium methyl sulphate and 0.32% propiconazole. In each of these cases the biocidal azole is present in an amount less than about 20% by weight of the combined biocidal azole and solvents-oils-emulsifiers.

U.S. Pat. No. 5,536,305 describes a wood preservative composition containing a 3-isothiazolone and optionally one or more other preservative compounds selected from propiconazole; tebuconazole; fenbuconazole; myclobutanil; azaconazole; iodopropargyl butyl carbamate; 2-thiocyanomethyl(thio)benzothiazole; chlorpyrifos; chlorothalonil; permethrin; dichlofluanid; cyfluthrin; cypermethrin; copper-8-quinolinolate; s-fenvalerate; bifenthrin; o-phenylphenol; dithiocarbamate compounds; copper naphthenate; zinc naphthenate; tributyltin oxide; pentachlorophenol; quaternary compounds; and the like; (b) a surfactant system consisting of at least one surfactant selected from the group consisting of sulfated anionics, sulfonated anionics, sulfosuccinated anionics, quaternary ammonium cationics, and amphoterics; and (c) at least one non-polar organic solvent.

U.S. Pat. No. 5,659,809 describes a concentrate package-mix composition for agricultural use comprising a water-soluble pesticide or plant growth regulator and a solid water-insoluble pesticide or plant growth regulator. The water-soluble pesticide or plant growth regulator is dissolved in a continuous aqueous phase and solid particles of the water-insoluble pesticide or plant growth regulator are suspended in the aqueous phase. These solid particles, individually or plurally, are intimately surrounded by a barrier layer, which comprises either one or both of a water-immiscible organic solvent or an emulsifying agent that has a hydrophile-lipophile balance (HLB) not greater than about 15. Among the soluble biocides listed include copper sulfate, and among the insoluble biocides listed include Tebuconazole. Examples of organic solvents which can be suitable in particular cases include water-immiscible aliphatic and aromatic hydrocarbons and hydrocarbyl alcohols, aldehydes and ketones, mono-, di- and trihydrocarbyl phosphates, silicone and siloxane oils, fatty acids and alkylesters and alkylamides thereof, and natural vegetable oils whether fractionated or not. This patent suggests for some compounds that both a cationic and an anionic emulsifier be used, and that one of these emulsifying agents, typically the cationic one, can be relatively hydrophilic or water-soluble, but it is important that the other, typically the anionic emulsifying agent, be lipophilic or of low water solubility.

U.S. Pat. No. 6,746,988 describes agricultural compositions comprising active ingredients and surfactant systems comprising alkyl polyglycosides, anionic surfactants selected from a polyarylphenol polyalkoxyether sulfate and a polyarylphenol polyalkoxyether phosphate, a basic compound, and optionally further comprising at least one nonionic surfactant. U.S. Pat. No. 6,746,988 claims agricultural compositions wherein the agriculturally active compound is a fungicide, an insecticide, an herbicide, a growth regulator, a safener, a plant activator, or a mixture thereof. The agriculturally active compound can be a fungicide such as for example bromoconazole, cyproconazole, epoxiconazole, fenbuconazole, fluqinconazole, hexaconazole, or tebuconazole. The agriculturally active compound can be an insecticide such as for example acrinathrin, bifenthrin, cyfluthrin, cypermethrin, permethrin, or tralomethrin. The basic compound can be a C8-18 alkyl amine polyalkoxylate, an alkanol amine, an alkanol amide, or a mixture thereof, and is preferably a tallow amine ethoxylate, a cocoamine alkoxylate, a oleylamine alkoxylate, a stearylamine alkoxylate, a linoleic diethanolamide, or a mixture thereof. The nonionic surfactant can be an ethylene oxide-propylene oxide block copolymer, ethylene oxide-butylene oxide block copolymer, a C₂₋₆ alkyl adduct of an ethylene oxide-propylene oxide block copolymer, a C₂₋₆ alkyl adduct of an ethylene oxide-butylene oxide block copolymer, a polypropylene glycol, a polyethylene glycol, a polyarylphenol polyethoxy ether, a polyalkylphenol polyethoxy ether, a polyglycol ether derivative of a saturated fatty acid, a polyglycol ether derivative of an unsaturated fatty acid, a polyglycol ether derivative of an aliphatic alcohol, a polyglycol ether derivative of a cycloaliphatic alcohol, a fatty acid ester of polyoxyethylene sorbitan, an alkoxylated vegetable oil, an alkoxylated acetylenic diol, or a mixture thereof. Exemplary C₈₋₁₈ alcohol polyethoxyether phosphates, carboxylates and citrates include STEPFAC™ 8180 (tridecylalcohol(EO)₃phosphate), STEPFAC™ 8181 (tridecylalcohol(EO)₆phosphate), and STEPFAC™ 8182 (tridecylalcohol(EO)₁₂phosphate), all from Stepan Corporation, Northfield, Ill., EMCOL™ CN-6 (tridecylalcohol(EO)₆carboxylate) from Cromptom Corporation, Greenwich, Conn., or BIOSOFT™ S100 (dodecylbenzene sulfonic acid) from Stepan Corporation, Northfield, Ill.

For insoluble biocides such as biocidal azoles, the cost of the emulsifying agents or solubilizing agents is a significant factor in the total cost, and therefore in the utility, of the resulting composition. Additionally, the inclusion of large quantities of surfactants in some formulations is believed to contribute to loss of active ingredient over time due to leaching thereof by water.

The formulation of products containing multiple active ingredients with very different physical properties is increasingly required in the marketplace to provide a broader spectrum of biological activity in a single product offering. The challenge involved in developing commercially acceptable products that contain a plurality of biocidal compounds, for both wood treatments and for agricultural uses, continues to increase due to the rapid emergence of more complex customer and regulatory requirements. These resultant products must exhibit excellent chemical stability and must also maintain a high level of physical stability under a severe range of storage and use conditions. Stability is critical to customer satisfaction and commercial success with a product since poor compatibility in the final use mixture can cause blockage of wood surfaces in wood preservation or blockage of sprayers in agricultural applications, preventing proper application of the product. At the same time, the number of inert chemicals approved for use in agrochemical and wood preservative compositions by the U.S. EPA has been decreasing due to more stringent requirements for toxicological and ecoogical data on these materials.

What is needed are new methods of formulating products, and the new resultant products, that comprise water-insoluble organic biocides (such as biocidal azoles) and that further comprise s approved inert auxiliary chemicals which have both robust performance characteristics and a good environmental safety profile, wherein said products are additionally useful when admixed with water-soluble products such as are typically encountered in agricultural products and with wood preservation products. What is further needed is a method of forming suspendible sub-micron particulates or droplets of tebuconazole or similar biocidal azoles, and an emulsifiable concentrate of tebuconazole or similar biocidal azoles and optionally with other biocides, said emulsifiable concentrate having greater than 20% by weight of the tebuconazole or similar biocidal azoles.

### SUMMARY OF THE INVENTION

In a first embodiment of the invention we present a concentrated emulsifiable formulation of tebuconazole and/or similar biocidal azoles which is a stable concentrate mixture having greater than 20%, preferably greater than 25%, more preferably greater than 30%, for example between about 30% and about 40% by weight of tebuconazole and/or similar biocidal azoles, that in use is compatible with a number of biocidal metal-containing formulations often encountered in agricultural use and in wood preservation use, including but not limited to aqueous suspensions of submicron basic copper carbonate or other sparingly soluble copper or zinc salts or oxides, and also with aqueous solutions of copper-amine complexes such as ammoniacal copper, copper monoethanolamine, and most preferably with copper monoethanolamine carbonate solutions such as those manufactured by the methods disclosed in U.S. Pat. Nos. 6,905,532; 6,905,531; and 6,646,147.

The concentrated emulsifiable formulation of tebuconazole and/or similar biocidal azoles advantageously includes A) an amide, B) a water-soluble non-ionic emulsifier, C) a water-insoluble anionic emulsifier, and D) a water-insoluble biocidal azole, wherein the concentrated stable emulsifiable formulation is a solution containing the water-insoluble biocidal azole in an amount greater than 20% by weight of the formulation.Preferably the amide is a compound of formula 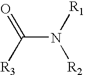
where R₁ and R₂ are independently H, a C₁-C₈ alkyl or alkenyl, a C₁-C₈ alkoxy, a C₁-C₈ alkene, a C₄-C₈ cyclic moiety or C₄-C₁₀ alkaryl, and R₃ is a C₁-C₁₂ alkyl or alkenyl, a C₂-C₁₂ alkoxy, a C₂-C₁₂ alkene, a C₄-C₈ cyclic moiety, a C₆-C₁₂ aryl or alkaryl, a C₆-C₂₀ alkyl ethoxylate or a C₆-C₁₄ fatty acid. The water-soluble non-ionic emulsifier is advantageously an alkyl ethoxylate, an alkenyl ethoxylate, an alkylphenol ethoxylate, an alcohol ethoxylate, a fatty acid ethoxylate, an EO/PO block copolymer, or mixture thereof, more preferably an ethoxylated C₁₂-C₂₄ vegetable oil with between about 20 and about 40 moles of EO per mole of oil. The water-insoluble anionic emulsifier is a metal or alkaline earth metal salt of benzosulfonate substituted in the ortho-, meta-, or para-position with C₈-C₁₆ alkyl or alkenyl, a C₁₀-C₁₈ alkoxy, or a C₈-C₁₆ alkene. The concentrated stable emulsifiable formulation may further include an N-alkyl-pyrrolidone, a non-azole organic biocide, or both.

The compositions of the current invention do not necessarily make a water-soluble formulation when admixed with water but rather form when the concentrated composition is admixed with water in a first embodiment an emulsion or in a second embodiment a sub-micron suspension. The emulsion concentrate is a stable solution that optimally contains 20% or more, preferably more than 25%, for example between about 30% to about 40% or alternatively between about 35% and 45% by weight of water-insoluble biocidal azoles including particularly tebuconazole dissolved in solvent and with one or more emulsifying agents. The emulsion concentrate, when diluted with a sufficient amount of water, forms an emulsion of very fine tebuconazole-solvent-emulsifier droplets.

In one embodiment of the invention the emulsion concentrate can be admixed with a concentrated solution comprising a copper amine complex such as ammoniacal copper, copper monoethanolamine, and the like prior to shipping and storing the resultant formulation. As used herein, when a copper-amine-containing solution is said to be “concentrated”, it means that the solution must be diluted with water to obtain a useful concentration for wood preservation. Alternatively, the emulsion concentrate can be kept as a separate concentrate for selling, shipping, and storing reasons, wherein the emulsion concentrate is mixed with a copper-amine solution at a time or point closer to the time and point of use, such as is described in the “Pack A” and “Pack B” formulations described in U.S. Pat. Nos. 5,527,384 and 5,634,967.

One advantage of the concentrate formulations of the present invention is that they contain a higher concentration of biocidal azole, such as tebuconazole, than did the concentrate formulations disclosed in the prior art, for example U.S. Pat. No. 5,527,384. The use of a more concentrated solution saves money and reduces solvent exposure. The ultimate users of the formulations of this invention are sometimes hesitant to use emulsion concentrates, as there is a very small amount of concentrate being added to large volumes of water, and there is concern that a significant portion of the concentrate may be trapped in “dead spots” or be plated out on equipment and such before a stable emulsion is formed. Loss of a small amount of concentrate can mean a significant reduction in the organic biocides present in the final treatment composition. There is also the requirement when admixing emulsion concentrates into aqueous solutions of high shear forces necessary to break particles/droplets into the desired sub-micron size. The preferred embodiments of the emulsion concentrates of this invention have a second advantage, however, which reduces or eliminates these concerns. The emulsion concentrates of the current invention are beneficially miscible when added in appropriate amounts to typical concentrated aqueous solutions of copper and amine, that is, in amounts that are normally used in wood preservation and in agricultural uses, and the resultant mixture forms emulsions only when further diluted to the strength useful in the agricultural applications and/or the wood preservation applications.

Current wood preservation technology uses water-soluble copper-amine complexes to provide the biocidally effective copper ions, in combination with at least one organic biocide. As is known in the art, generally a concentrated solution of copper and an amine includes solutions having greater than about 8%, typically greater than about 10%, usually between 10% and 15% by weight copper, and between 3.5 and 4.5 equivalents of amine per mole of dissolved copper. To reduce shipping and storage costs, manufactures ship the water-soluble copper complexes as a concentrate of, for example, aqueous copper/monoethanolamine/carbonate which typically has 9%-13% copper, 31%-45% monoethanolamine, 7%-12% carbonate, and 53% to 30% water. While less water (that is, a more concentrated solution) is generally considered better due to economics of shipping and storing the copper amine concentrate, such aqueous formulations of copper amine can be inexpensively produced and can exhibit greater handle-ability and stability over a range of environmental conditions than water-free products. Other compositions used in for example wood treatment or in agricultural uses where a copper-amine complex is desired have similar compositions, but some or all of the carbonate may be replaced by other ions such as borate, some or all of the monoethanolamine may be replaced by other alkanolamines, alkyl amines, or ammonia, and some or all of the copper may be replaced by for example zinc. Such changes may substantially increase or decrease the amount of copper, water, amine, and anions present, but the invention is useful in many of these embodiments nevertheless.

To treat wood using a soluble copper amine and tebuconazole such as is described in for example U.S. Pat. No. 5,527,384, a very dilute composition of about 0.1% to about 1.5% copper, more typically between about 0.2% and about 0.5% of copper, and only about 0.004% to about 0.06%, more typically about 0.008% to about 0.02% of tebuconazole is needed. As discussed in the background section, the concentrate formulations used with copper-amine formulations that were described in for example U.S. Pat. No. 5,527,384 contained 20% or less by weight of tebuconazole or other biocidal azole relative to the amount of oils, solvents, and emulsifiers present. Using a 20% by weight tebuconazole “Pack B” such as described in U.S. Pat. No. 5,527,384, along with a typical copper monoethanolamine carbonate formulation having 10% copper, to formulate 100 parts of a “typical” wood treatment composition having 0.2% copper and 0.008% tebuconazole would require admixing 2 parts of the copper monoethanolamine concentrate, 0.02 parts of the Pack B concentrate, and about 98 parts of water. An operator might be concerned that the small amount of tebuconazole added might not be appropriately distributed through-out the wood treatment formulation, and/or that the small amount of organic biocide-containing material might not encounter sufficient shear forces to provide small enough droplets that the emulsion can both be injected into wood and be sufficiently distributed throughout the wood.

Surprisingly and advantageously, however, preferred emulsion concentrates of this invention can first be admixed with a copper- and amine-containing concentrate, and the emulsion concentrate will appear to be miscible therein. The emulsion is formed as the additional water is added to bring the composition to the desired treatment strength. Therefore the operator or end-user can be more certain that substantially all of the biocidal azole is distributed throughout the end-use agricultural formulation and/or wood treatment formulation. The emulsion concentrate when first admixed into a concentrated aqueous copper amine solution, such as concentrated aqueous copper/monoethanolamine/carbonate, becomes pre-diluted and pre-dispersed, and therefore on dilution with water more easily forms small, typically submicron sized droplets with less shear forces.

In an important embodiment this concentrated emulsifiable formulation of biocidal biocidal azole comprises or consists essentially of the following components:

A) a basic component, for example an amine, preferably an amide of formula 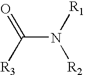
where R₁ and R₂ are independently H, a C₁-C₈ alkyl or alkenyl, a C₁-C₈ alkoxy, a C₁-C₈ alkene, a C₄-C₈ cyclic moiety or C₄-C₁₀ alkaryl, and are preferably independently a C₁-C₄ alkyl, most preferably a methyl moiety, and R₃ is a C₁-C₁₂ alkyl or alkenyl, a C₂-C₁₂ alkoxy, a C₂-C₁₂ alkene, a C₄-C₈ cyclic moiety, a C₆-C₁₂ aryl or alkaryl, a C₆-C₂₀ alkyl ethoxylate (as used herein the term ethoxyate means there are a plurality of ethoxy (EO), propoxy (PO), or both, but preferably EO, subunits polymerized or condensed into a chain) or a C₆-C₁₄ fatty acid, and is preferably a C₄-C₁₀ alkyl, more preferably a C₆-C₈ alkyl;

B) a borderline water-soluble non-ionic emulsifier, advantageously selected from an alkyl ethoxylate, an alkenyl ethoxylate, an alkylphenol ethoxylate, an alcohol ethoxylate, a fatty acid ethoxylate, or mixture thereof, preferably an ethoxylated C₁₂-C₂₄ vegetable oil such as an ethoxylated castor oil with between about 10 and about 50, preferably with between about 20 and about 40, for example between about 25 to about 35 moles of EO per mole of oil, and/or optionally but less preferably an EO—PO or EO-butylene oxide block copolymer;

C) a water-insoluble anionic emulsifier, for example an alkaline earth salt of an alkyl benzosulfonate emulsifier of formula: 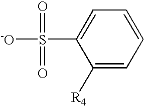
where the sulfonate and R4 groups can be ortho-, meta-, or para- to one another, and where R4 is a C₈-C₁₆ alkyl or alkenyl, a C₁₀-C₁₈ alkoxy, or a C₈-C₁₆ alkene, preferably a C₁₀-C₁₄ alkyl; and

D) a water-insoluble organic biocidal azole, for example tebuconazole, propiconazole, cyproconazole, tetraconazole, difenoconazole, diniconazole, penconazole, imibenconazole, propiconazole, azaconazole, epoxyconazole, fenbuconazole, fluqinconazole, hexaconazole, imibenconazole, ipconazole, metconazole, penconazole, triticonazole, fluquinconazole, or mixture thereof, most preferably comprising or consisting essentially of tebuconazole.

Of the component (A) compounds, amides are preferred, and one or more of N,N-dialkylcaprylamide, N,N-dialkylcapramide, N,N-dialkyloctanamide, N,N-dialkyldecanamide, N-alkylcaprylamide, N-alkylcapramide, N-alkyloctanamide, N-alkyldecanamide, caprylamide, octanamide, capramide, and decanamide are particularly preferred, where the alkyl groups independently comprise between one and three carbon atoms. N,N-dimethylcapramide and mixtures N,N-dimethylcapramide and N,N-dimethylcaprlyamide are particularly preferred as it is commercially available, inexpensive (˜$1.30 per pound versus for example about $4.00 per pound for N-methylpyrollidone), and are an EPA-approved inert ingredient exempt from tolerance according to 40CFR 180.1001. Many commercially available blends of the amide contain various fractions of N,N-dimethylhexanamide, N,N-dimethyloctanamide, and/or N,N-dimethyldecanamide, more typically approximately 50/50 by weight blend of N,N-dimethylcaprylamide and N,N-dimethylcapramide. N-alkyl-pyrrolidones can be used as a component (A) compound, including for example N-heptyl-2-pyrrolidone or with some adjustment to the other emulsifiers even N,N-dimethyl-pyrolidone can be used, but at a significant additional cost. Advantageously, if alkyl-pryrolidones are included in the component (A) compounds then the alkyl-pryrolidones are present in an amount lower than the amount of amide.

The upper limit on the amount of component (A) compounds is more an economic limitation as opposed to a physical limitation. Advantageously the emulsion concentrate has less than 4 parts of component (A) compounds per part of biocidal azole. While a composition comprising more than 4 parts of component (A) compounds per part of tebuconazole can be used, such use is wasteful, adding excess cost to the formulation with little benefit. Advantageously there is between 1 part and 3 parts, preferably between 1.3 parts and 2 parts, for example between about 1.5 parts and 1.8 parts of component (A) per part of biocidal azole. Advantageously there is between 1 part and 3 parts, preferably between 1.3 parts and 2 parts, for example between about 1.5 parts and 1.8 parts of component (A) amides per part of tebuconazole and/or similar biocidal biocidal azoles.

Useful component (B) compounds include high molecular weight esters of natural vegetable oils such as the alkyl esters of oleic acid. Of the component (B) non-ionic emulsifiers, water-insoluble ethoxylated castor oil having about 15 to about 40 moles of EO per mole of oil is preferred, castor oil having about 25 to about 35 moles of EO per mole of oil is more preferred, and castor oil having 30 moles of EO per mole of oil is particularly preferred. Another preferred component B compound is an ethoxylated tri(C6-C14 alkyl)alcohol having 5 to 30 EO per mole of alcohol, in particular a tridecylalcohol POE (12). A variety of ethoxylated vegetable oils should work, for example soybean oil POE 30, but the cost is usually higher than ethoxylated castor oil or ethoxylated tridecylalcohol. The higher the degree of ethoxylation the greater the water solubility and the higher the resultant hydrophile-lipophile balance. To get good emulsification, you need reasonably low water solubility or you will get separation, and you need to match the emulsifier hydrophile-lipophile balance to the solvent/A.I. hydrophile-lipophile balance. Castor oil having 60 moles of EO per mole of oil is not recommended, as the surfactant is water soluble and the surfactant and tebuconazole will separate when an emulsion is formed, causing the tebuconazole to crystallize. Castor oil having 60 moles of EO per mole of oil can be added to the formulation, but on admixing with water to form an emulsion mush of this water-soluble material will be leached from the emulsified biocidal droplets, and the stabilizing contribution of this material will be significantly reduced. Of course, if submicron particles of a water-insoluble azole are desired for some reason, they can be produced in this manner, that is, using more soluble component (B) compounds.

The upper limit on the amount of component (B) compounds is more an economic limitation as opposed to a physical limitation. Advantageously the composition comprises less than 1 parts of component (B) compounds per part of water-insoluble biocidal azole. While a composition comprising more than 1 parts of component (B) compounds per part of water-insoluble biocidal azole such as tebuconazole can be used, such use is wasteful, adding excess cost to the formulation with little benefit. Advantageously, for admixing the concentrate into slurries or solutions of copper/zinc salts and/or oxides in water, there is between 0.03 parts and 0.3 parts, preferably between 0.08 parts and 0.2 parts, for example between about 0.1 parts and 0.15 parts of component (B) non-ionic emulsifiers per part of tebuconazole and/or similar water-insoluble biocidal azoles. Advantageously, for mixing the concentrate into aqueous copper amine solutions, there is between 0.08 parts and 0.4 parts, preferably between 0.1 parts and 0.3 parts, for example between about 0.11 parts and 0.15 parts of component (B) non-ionic emulsifiers per part of tebuconazole and/or similar biocidal azoles.

In less preferred embodiments, the component (B) compounds can be partially or fully replaced by alkyl polyglycosides as are described in U.S. Pat. No. 6,746,988, having a C₆-C₃₀ monovalent organic radical, between 2 and 40 divalent alkylene radicals such as ethylene, and between 1 and 6 saccharide residues each having 5 or 6 carbon atoms such as glucose. The glucose may react with copper(II) ions and reduce them to copper(I) ions over time, which can reduce the biocidal efficacy of the copper.

Of the component (C) anionic emulsifiers, water-insoluble alkyl benzosulfonate emulsifier are preferred, but alkyl benzosulfates and alkyl benzophosphates having similar numbers of carbon atoms are also useful. Alkyl benzocarboxylates are less preferred. There can be two sulfonate moiteies, phosphate moieties, sulfate moieties or combinations thereof on the benzene ring, but generally only one such polar moiety is needed. C₁₀-C₁₄ alkyl benzosulfonate emulsifiers are preferred, but C₁₀-C₁₄ alkyl ether phosphates, C₁₀-C₁₄ alkyl ether carboxylates, C₁₀-C₁₄ alkyl ether sulphates, C₁₀-C₁₄ alkyl phenol ether phosphates, and C₁₄-C₁₈ fatty alcohol sulphates can be used in less preferred embodiments, particularly if admixed with a preferred C₁₀-C₁₄ alkyl benzosulfonate emulsifier. As used herein, a “C₁₀-C₁₄ alkyl benzosulfonate” has a benzene moiety, a sulfonate moiety, and a C₁₀-C₁₄ alkyl moiety. Alkaline earth salts, typically calcium salts, are preferred as the alkali salts have too much solubility in water. Calcium dodecylbenzenesulphonate is the most preferred component (C) anionic emulsifiers.

Advantageously, for admixing the concentrate into slurries of copper/zinc salts and/or oxides in water, there is between 0.02 parts and 0.2 parts, preferably between 0.03 parts and 0.1 parts, for example between about 0.04 parts and 0.06 parts of component (C) anionic emulsifiers per part of tebuconazole and/or similar biocidal azoles. Advantageously, for mixing the concentrate into aqueous copper amine solutions, there is between 0.03 parts and 0.3 parts, preferably between 0.04 parts and 0.2 parts, for example between about 0.06 parts and 0.1 parts of component (C) anionic emulsifiers per part of tebuconazole and/or similar biocidal azoles.

The upper limit on the amount of component (C) anionic emulsifiers is more an economic limitation as opposed to a physical limitation. Generally, subject to the limitation that the resultant emulsion concentrate comprise more than 20%, preferably more than 25% by weight of biocidal azole, the above-described formulation in a preferred embodiment comprises 1.3 parts and 2 parts of component (A) amides, between 0.1 parts and 0.3 parts of component (B) non-ionic emulsifiers, and between 0.04 parts and 0.2 parts of component (C) anionic emulsifiers per part of tebuconazole and/or similar biocidal azoles. The amount of component (B) compounds can beneficially range in an amount greater than 0.3 parts of component (B) per part of tebuconazole and/or similar biocidal azoles, for example between about 0.4 parts and 2 parts of component (B) per part of tebuconazole and/or similar biocidal azoles, and the amount of component (C) compounds can beneficially range in an amount greater than 0.3 parts of component (C) anionic emulsifiers per part of tebuconazole and/or similar biocidal azoles, for example between about 0.4 parts and 1.5 parts of component (C) anionic emulsifiers per part of tebuconazole and/or similar biocidal azoles. This greatly increased amount of component (B) and component (C) compounds can reduce and even eliminate the amount of component (A) basic material needed. Such replacement may not be economically feasible unless the amount of solvents must be reduced or eliminated for other reasons such as regulations. Increasing the amount of component C to between 0.5 parts to 1.2 parts per part of tebuconazole and/or similar biocidal azoles can eliminate the need for a component (A) basic material, and at least a portion of the component (B) non-ionic emulsifiers can be replaced with relatively inexpensive substitutes such as an EO—PO or EO-butylene oxide block copolymer. Such changes can provide an emulsion concentrate that has a greater working range in terms of water hardness.

The surfactant systems, agricultural compositions, concentrates, and polymer-stabilized aqueous emulsions of the invention may further comprise other additives, such as pigments, UV-protectorants, fire-retardant polymers, waxy polymers, thickeners, flow enhancers, wetting agents, antifoaming agents, buffers, chelators, deposition enhancers, adjuvants, evaporation retardants, frost protecting agents, and the like. The composition can comprise other emulsifiers, surfactants, moldicides, and the like.

The emulsifier concentrates can be formulated merely by mixing the (A), (B), (C), and (D) ingredients, or alternatively by mixing the (B), (C), and (D) ingredients. The resulting mixture is gently stirred or agitated to give a concentrate. If one or more of the ingredients are solids, it may be necessary to heat a slurry of the ingredients to form a stable composition in a short period of time. Alternatively, when a biocidal compound having a melting point above 25° C. is used, concentrate compositions can be prepared by dissolving the biocidal compound in the organic solvents to form an liquid phase. The surfactants can then be added. If a surfactant is in paste or solid form, it is preferred to premelt the surfactant prior to mixing it with the other compounds. Alternatively, if the solid wood preservative compound is heat stable, all the components may be added together in a single vessel and the vessel heated sufficiently to form the microemulsion concentrate. The latter method has the advantage that it is a one-step addition.

The invention includes a method of treating wood. In a simple method, water, a sufficient amount of a water-soluble or water dispersible copper-containing composition, and the emulsifier concentrate (including the biocidally active agent such as tebuconazole) are admixed with sufficient shear that a stable formulation having the tebuconazole or other azoles in a stable emulsion. The resultant emulsion is sufficiently stable for use in either soluble copper amine formulations or in slurries of suspended sub-micron sparingly soluble copper salts such as a submicron sparingly soluble basic copper carbonate salt having its normal dispersants. Alternatively, the emulsifier concentrate (including the biocidally active agent such as tebuconazole) can be admixed with a copper-amine concentrate, and then water is added with only moderate shear necessary to form the stable formulation having the tebuconazole and/or other azoles in a stable emulsion.

As mentioned above, it is known that combinations of organic biocides are useful for a variety of uses. Indeed, while copper ions have formed the basis for most commercially feasible wood preservative formulations, where more than 80% by weight (and typically between 95% and 99% by weight) of the biocidal material has comprised copper, the amount of copper ions (and/or other inorganic salts/oxides such as zinc borate, copper borate, copper hydroxide, copper carbonate, copper oxides, and the like) can significantly reduced or eliminated if an effective combination of organic biocides is used. The use of combinations of organic biocides, each effective in a biocidally effective amount, as opposed to a single organic biocide reduces the possibility that a pest may develop a tolerance and be able to attack treated wood. Indeed, a principal activity of copper ions in traditional wood preservation systems can be said to be merely imparting sufficient biocidal activity to discourage the growth of those few pests that are resistant to the organic biocides, that is, to protect the organic biocides within the wood matrix. A variety of patents describe synergistic interactions (or at least effective biocidal activity) between certain biocidal azoles. Generally, combinations of tebuconazole, propiconazole, azaconazole have desirable properties. Addition of at least one non-azole biocide having a different active component can be useful. Early patents described admixing biocidal azoles with a biocidal quaternary ammonium compound. A wide variety of patents have disclosed effective combinations with other organic biocides.

The use of combinations of organic biocides can open the door for a reduced-copper or a copper-free wood preservative treatment. Such a treatment can be said to contain less than 80% by weight of inorganic copper salts or oxides, zinc salts or oxides, and/or borates, compared to the total weight of biocidally effective material in the wood preservative system. Preferably such reduced-copper wood preservative compositions comprise between 10% and 79%, typically between 10% and 50%, by weight of inorganic copper salts or oxides, zinc salts or oxides, and/or borates, compared to the total weight of biocidally effective material in the wood preservative system. Copper-free wood preservative systems comprise between 0% and 10%, typically less than 5%, by weight of inorganic copper salts or oxides, zinc salts or oxides, and/or borates, compared to the total weight of biocidally effective material in the wood preservative system.

Preferred compositions of the invention comprise a biocidal azole. The composition of the invention may include or alternatively may be free of isothiazolones such as described for example in U.S. Pat. No. 5,536,305. The composition of the invention may include or alternatively may be free of imidazolinones such as described for example in U.S. published application 20020137783.

Such combinations are useful to eliminate certain environmental concerns having to do with metals leaching in certain environments, but such reduced-copper and copper-free formulations generally require a higher concentration of organic biocides than does traditional copper-based formulations, and are therefore even more susceptible to the high costs of surfactants, emulsifiers, and the like needed to place the organic biocides in condition to be injected into wood (as either solubilized biocide, as a sub-micron emulsion of biocide, as a milled organic biocide, or as any combination thereof). Such a reduced-copper or copper-free formulations are even more impacted by the high costs of surfactants and emulsifiers, especially when the amount of active ingredients is below about 25%. And typically, in the prior art formulations biocidal azole concentrates are present in amounts lower than 10%.

Herein is disclosed a method for preparing highly loaded emulsifiable concentrate formulations of various organic biocides and combinations thereof which may find similar utility as wood preservative compositions. Again, the formulations advantageously are a stable composition comprising greater than 25%, preferably greater than 30%, for example between 35% and 45% by weight of one or more biocidal triconazoles, and greater than 0.2%, for example greater than 0.5%, or alternatively greater than 1%, for example between about 1% and about 5%, of a non-azole sparingly soluble or insoluble (in water) organic biocide. Permethrin, cypermethrin, cyfluthrin, bifenthrin and imidacloprid are examples of useful non-azole insecticides that could be used (especially in combination with one or more biocidal azoles in an emulsion concentrate) in wood. Others that could be used in combination with one or more biocidal azoles in an emulsion concentrate include indoxacarb, tetramethrin, fipronil and cyhalothrin. Any combinations of biocidal azoles can be used, though tebuconazole and/or propiconazole are most preferred. Generally, the biocidal azoles are present in an amount between about 2 to about 100 parts by weight, for example between about 4 parts and about 50 parts by weight, per part of non-azole biocide. Any combinations of these compounds, and optionally including one or more added moldicides and/or fungicides, can be used in an emulsion concentrate to treat wood either alone, in a copper-free formulation, in a reduced-copper formulation, or in a traditional (copper based) wood treatment formulation.

For example, Arch Wood Protection recently received US EPA registration on an end use wood preservative product having 5.0% tebuconazole, 5.0% propiconazole, 0.5% imidacloprid, and 89.5% inert ingredients (surfactants, etc). This is near the solubility limit of the carrier, that is, very little additional biocidal azole can be dissolved in that material. This is about 20 parts biocidal azoles per part of imidacloprid. Other commercially importants formulations include tebuconazole and imidacloprid in a ratio of about 50 parts to 1 part by weight, and 4 parts biocidal azoles (tebuconazole and propiconazole) per part by weight of permethin. In contrast, an exemplary formulation of the present invention comprises 15.0% tebuconazole (range 1-35%, preferably 5-20%), 15.0% propiconazole (range 1-40%, preferably 5-20%), 1.5% imidacloprid (range 0.2%-3%), 5.0% of an emulsifier blend (range 1-15%), 60.0% of a basic solvent such as N,N-dimethyldecanamide/octanamide (range 0.0-65%), and 3.5% N-methyl pyrrolidone (range 0.0-50%). The alkyl pyrrolidones can be included in component (A) compounds if an organic biocide is particularly difficult to dissolve. Imidacloprid is sparingly soluble in most every EPA approved solvent, but advantageously NMP is used to dissolve greater than 1%.

An emulsifier blend which has been found to be effective is a mixture of:

1) an essentially water insoluble anionic surfactant such as calcium dodecylbenzene sulfonate, linear or branched (or similar alkyl benzosulfonates as described herein);

2) a water soluble surfactant such as EO—PO butyl block copolymer or variation thereof (as described herein); and

3) a water soluble surfactant such as an alcohol ethoxylate, namely octyl, nonyl, decyl, and/or tridecyl alcohol with 2-25, preferably with 3-15, moles of ethylene oxide (or variants as described herein). Advantageously the alkyl benzosulfonates are present in an amount between 10% and 45%, preferably between 25-35% Calcium DDBSA (available commercially as Ninate 60E™, from Stepan Corp). Advantageously the block copolymer (or variations thereof) is present in an amount between about 5% and 50%, preferably between 10-30% EO—PO butyl block copolymer (available commercially as Toximul 8320™, from Stepan Corp). Advantageously the alcohol ethoxylate (or variations thereof) is present in an amount between about 15% and 70%, preferably between 30-60% Tridecyl alcohol POE-12 (available commercially as MAKON TD-12™ by Stepan Corp.). Those skilled in the art (with the benefit of this disclosure) could devise a number of blends of these and other surfactants which would sufficiently emulsify such compositions. Other solvents that may be effective in dissolving these particular biocides include 2-ethylhexanol, Aromatic 100, 150 and so on.

Other biocides which could be used in this invention include those in the azole family and other organic molecules in general (fungicides and insecticides) that are applicable to wood preservation. These could be combined in any number of mixtures and variations, including two-ways, four-ways, etc. or alone. A large number of surfactants that can be used as emulsifier components to make an EC. It really comes down to the formulator's preference. Similarly, there are many other solvents that could be used to dissolve these active ingredients and make an EC or microemulsion from an emulsion concentrate (MEC). When the compositions are diluted and undergo sufficient shear to form a microemulsion, the resultant microemulsion is stable at all reasonably useful levels of water dilution and remain thermodynamically stable and clear, opalescent, or only slightly cloudy. Tebuconazole and propiconazole are very soluble in N,N-dimethylcapramide (“N,N-DMC”) and thus the economics are favorable for compositions using that solvent or a close variant thereof, e.g., octanamide. Other useful solvents include fatty acid methyl esters (i.e. methyl soyate), Aromatic 100, 150 & 200, 2-ethylhexanol, etc.

The benefits of the invention are; 1) cost savings in making a much more highly concentrated formula; 2) cost savings due to use of relatively inexpensive components; and 3) potentially reduced leaching levels compared to other compositions which may consist primarily of water soluble carriers.

The concentrates are used to form an emulsion of the biocides in an aqueous composition. For preserving wood, the resultant emulsion is then injected into wood using any of the pressure processes known in the art.

We believe the agricultural compositions and wood preservation compositions of the invention may be mixed with water to form stable suspoemulsions. Suspoemulsions have a liquid aqueous phase, which is generally a continuous phase, a liquid oil phase, which is generally a discontinuous phase dispersed in the aqueous phase, and a solid particulate phase, which is a discontinuous phase dispersed in one or both of the liquid phases.

Alternatively, solid sub-micron particles of solid tebuconazole can be generated, suspended, and be injected into wood. We have recently discovered that smaller amounts of surfactant can be used, per part of tebuconazole, to form a stable slurry wherein the particles comprise sub-micron tebuconazole. These stable sub-micron slurries of tebuconazole have less than 4 parts surfactant, preferably less than 2 parts surfactant, for example between about 0.1 and about 1 part, by weight total of surfactants and dispersants per part of tebuconazole.

A preferred injection procedure for injecting biocidal material into wood includes one or more of the following four steps:

1) At least partially drying the wood, for example drying to remove at least 30%, preferably at least 50%, of the total moisture that can be removed by air drying the wood in ambient conditions. Green wood comprises sufficient air volume that a sufficient amount of wood preservative can be injected, but a more concentrated slurry would be required as compared to injecting into (at least partially) dried wood. Additionally, penetration through the wood would be lower with green wood than with at least partially dried wood.

2) Subject the wood to vacuum, e.g, to below about 0.5 atmospheres absolute pressure prior to injecting the slurry or emulsion, and/or subject the wood to pressurized carbon dioxide, e.g., above about 30 psig, then vent the wood to atmosphere, prior to injecting the slurry or emulsion. When slurry or emulsion is injected into wood, air in the wood is compressed. If no vacuum and/or carbon dioxide exposure is used, then the air in the wood will be compressed to ˜one tenth of its original volume (generally wood treatment pressure is lower than 150 psi) which will typically be in the center of the wood, and the slurry will therefore not reach the center one tenth of the wood. Further, releasing pressure causes the air to expand and push a portion of the injected fluid out from the wood, and this expelled fluid may contain some of the emulsion, solution, or biocidal particles that make up the wood preservative. A vacuum of as low as one half an atmosphere will reduce the amount of wood the slurry will not penetrate from one tenth to one twentieth of the total wood volume, and on releasing the pressure much less of the injected fluid will be expelled by the expanding air. In practice, even more air might be swept from the wood as water in the wood is vaporized and escapes the wood. Alternatively or in addition to the vacuum process, injecting carbon dioxide into the wood and then venting this carbon dioxide to atmospheric pressure prior to injection will cause a portion of the air in the wood to be replaced by carbon dioxide. Carbon dioxide is so soluble in the slurry that it acts much like a vacuum, in that the carbon dioxide once dissolved in the water will not be compressed and will not keep slurry from being injected into wood.

3) Inject the injectable aqueous slurry or emulsion into the wood by immersing the wood in the slurry and then exerting an injection pressure of from above atmospheric pressure to about 300 psi, typically between about 75 psi and 150 psi. The pressure is then maintained for a period of time that can range from a few minutes to many hours, and then the pressure is released. The drier the wood is made in step 1 prior to injection and the more rigorous the vacuum and/or carbon dioxide exposure is in step 2, the less time is needed where pressure should be maintained. If using 150 psi injection pressure on wood having less than half of the water originally in the green wood, and also being exposed to sufficient vacuum and/or carbon dioxide cycles to remove 90% of the air in the dried wood, then the pressure maintenance period can usually be reduced to between 2 and 15 minutes (depending on the thickness of the wood being treated).

4) At least partially dry the wood, to further fixate the injected biocidal materials and/or particles into the wood matrix.

We have described forming emulsion concentrates which comprise greater than 25% biocidal azoles that is dissolved into the components making up the emulsion concentrate. We have found that 45% is the maximum amount of biocidal azole that can be formulated into a useful stable emulsion concentrate. In contrast, milled tebuconazole can have a substantially or completely solid phase of one or more biocidal azoles, and may also comprise non-azole biocides, especially those listed as useful for the emulsion concentrate formulations. One additional formulation is milled sub-micron biocidal azole particles (with or without any non-azole biocides) contacted with between 0.05 parts and 2 parts, for example, of the solvating components of the emulsion concentrate. The resulting composition will have many properties when diluted with water of an emulsion, but particles may have a submicron particle comprising a solid phase of the biocide(s) within the emulsion droplets, thereby allowing greater than 40% by weight of active ingredients.

One problem with an “all organic” biocidal formulation is that the analyses needed to demonstrate an active amount of material is present in wood is made much more costly and time-consuming. For this reason, it is advantageous to incorporate into a treatment one or more pigments or readily analyzed compounds (typically containing a metal such as iron or copper) wherein the readily analyzed compound follows (is formulated with and remains with) the organic biocides. Organic metal compounds (organometallic compounds), and particularly organic copper compounds, such as copper oxime, can fulfill that role if used in small quantities, e.g., less than the amount of biocidal azoles, for example less than 1 part of organometallic compound per part of biocidal azole. Such material is able to be formulated into the milled solid biocide and/or into an emulsion concentrate, but is readily analyzable by methods known to the wood industry. An alternative option is to add copper partially or completely replacing the calcium in the alkyl benzosulfonate or similar emulsifier, provided the copper remains with the emulsifier. The amount of copper will be a trace, and leaching problems would be negligible, but it is relatively easy to test wood even for trace levels of copper.

As used herein, unless explicitly stated, percentages are percent by weight and parts are parts by weight. All patents mentioned herein are incorporated by reference for all allowable purposes.

### EXAMPLES

### Example 1

A total of 20 g of tebuconazole technical (98%), 31 g of N,N-dimethylcaprylamide solvent, and 3 g of emulsifier blend (70% castor oil POE 30 plus 30% CaDDBS) were admixed in a laboratory blender. This is equivalent to adding 0.59 parts of tebuconazole technical to 1 part by weight of a biocidal azole-emulsifier formulation comprising about 91.1% N,N-dimethylcaprylamide, about 6.2% castor oil POE 30, and about 2.6% CaDDBS. Alternatively, this composition can be said to contain 1.58 parts of N,N-dimethylcaprylamide, 0.107 parts of castor oil POE 30, and 0.046 parts of calcium dodecylbenzene sulfonate (CaDDBS) per one part of tebuconazole. The resultant clear, homogeneous mixture generates a stable emulsion upon dilution in water and is compatible with concentrated copper/monoethanolamine/carbonate formulations and with basic copper carbonate suspensions. Injection of an emulsified biocidal azole into wood could be expected to reduce biocidal azole leaching from the wood matrices, when compared to for example biocidal azoles that are solublized in water by large quantities of soaps or such prior to injection into wood.

### Example 2

Certain changes can be made to obtain a more hard-water-tolerant emulsion concentrate. In a mixer a biocidal azole-emulsifier formulation was prepared using 49.7% tridecylalcohol POE (12), 20% butyl block copolymer (a.k.a. Tergitol XD or Toximul 8320), and 30.3% of a branched calcium dodecylbenzene sulfonate (60% active). The resulting biocidal azole-emulsifier formulation had a capacity to solubilize the biocides in a concentrated emulsion concentrate which when admixed with large quantities of water such as would be used to treat wood forms an emulsion similar to the above-described example. This second formulation, however, also gave improved emulsification and hard water compatibility.

### Comparative Example 3

Milling tebuconazole with 0.3 mm zirconium oxide milling material. Tebuconazole is a preferred organic biocide for use in wood and also for selected foliar applications and for incorporation into biocidal materials. Tebuconazole is difficult to mill. Exemplary other preferred biocides from the same class of material include cyproconazole, tetraconazole, difenoconazole, diniconazole, penconazole, imibenconazole, propiconazole, azaconazole, and epoxyconazole. These are also believed to be difficult to mill. A CB Mills Vertical Mill Model: L-1 mill was used in this experiment. A tebuconazole suspension concentrate was made that contained about 20.5% (by weight) Tebuconazole, 3% of a Block Copolymer dispersant/stabilizer, 1.5% of a Napthalene Sulfonate dispersant/stabilizer, and 0.03% of an organopolysiloxane dispersant/stabilizer in water. The original d₅₀ (the diameter at which 50% by weight of the particles have an effective diameter equal to or less than the d₅₀) of the tebuconazole, prior to wet milling, was about 25 microns, with about a fourth of the tebuconazole in particles having a diameter greater than only about 4% by weight having a particle size less than 4 microns. The milling media was 0.3-0.5 mm Yttrium doped Zirconium Oxide. The density of the yttrium-doped zirconium oxide is ˜6.0 g/cm³. Milling for about 2.5 hours produced a stable suspension having the following particle size distribution: 96.4 weight % having a diameter of 10 microns or less, 92.2 weight % having a diameter of 1 micron or less, and 71.5 weight % having a diameter of 0.2 microns or less. This is not a particularly good slurry for injecting into wood. Generally, for injecting into wood, we prefer less than 2% by weight of particles have a diameter greater than 1 micron, more preferably 0.7 microns. The residual large particles can be separated from the slurry by centrifugation and then be sent back to the mill for additional milling.

TABLE 1
Wet ball milling Tebuconazole with 0.3 mm zirconia

| Milling | | | | | | |
|---|---|---|---|---|---|---|
| Time | Particle Size Data - Volume % With Diameter | | | | | |
| Mins. | >50 μm | 25-50 μm | 10-25 μm | 1-10 μm | 0.2-1 μm | <0.2 μm |
| 0 | 26.6 | 27.2 | 42.2 | 4 | — | — |
| 150 | 0 | 0 | 3.6 | 4.2 | 20.7 | 71.5 |

The above-described composition does not have a particle size distribution which will result in a commercially acceptable injectable wood composition. The composition can be further treated with for example a centrifugal finishing technique which effectively removes all particles with an effective diameter greater than 2 microns to form an injectable composition—a technique removing all particles greater than 2 microns will remove most particles with a size over 1 micron and a substantial fraction, typically 10% to 50%, of particles over about 0.7 microns.

### Example 4

It is often necessary to add an alkyl pyrrolidone as a part of the component (A) compounds to formulations containing biocides that are very difficult to solubilize. Such a highly loaded emulsifiable concentrate formulation for use with various organic biocides and combinations thereof and which find utility as wood preservative compositions was made by 1) admixing 15 parts tebuconazole, 15 parts propiconazole, and 1.5 parts imidacloprid in a dissolver; 2) adding 5 parts of an emulsifier blend (70% castor oil POE 30 plus 30% CaDDBS), 60 parts of a mixture of N,N-dimethyldecanamide and N,N-octanamide, and 3.5 parts of N-methyl pyrrolidone. The composition was mixed and formed a stable emulsion concentrate which when added to water readily formed a micro-emulsion suitable for injecting into wood.

### Example 5

A highly loaded emulsifiable concentrate formulation for use with various organic biocides and combinations thereof and which find utility as wood preservative compositions was made by admixing 21.3 parts of technical tebuconazole (97% active, or 20.6 parts tebuconazole), 65.7 parts of a mixture of N,N-dimethylcapramide and N,N-dimethylcaprylamide, 4 parts of tallowamine 7.0 (can substitute cocoamine 7.0 but cocoamine is more expensive), and 9 parts of an emulsifier blend containing 30% CaDDBS, 20% of a block copolymer, and 50% tridecyl POE 12. The tallow amine and/or CAM coco amine assist in compatibilizing the emulsion when mixed with dilute copper MEA carbonate solutions, and provides added insurance that the tebuconazole won't precipitate out over time from the pressure treatment solution if there are interruptions in the treatment process.

### Example 6

The solvents and emulsifiers described herein are also particularly useful for preparing emulsion concentrates of other very-difficult-to-solubilize biocides, such as abamectin (also known as avermectin) and halogenated and non-halogenated isothiazolones, for example a 3-isothiazolone having a C₄ to C₁₂ alkyl substituent, or 4,5-dichloro-2-n-octyl-4-isothiazolin-3-one. These biocides can also be used in lower quantities in the azole formulations discussed herein.

A stable abamectin microemulsion concentrate useful in wood treatment and in agriculture was prepared by admixing 1.83 parts of abamectin technical (98.2%), 20 parts of a mixture of N,N-dimethylcapramide and N,N-dimethylcaprylamide, 10 parts of propylene glycol (antifreeze), 15 parts of castor oil POE 36 (Toximul 8242™ from Stepan Corp.), 20 parts of a butyl block copolymer (Toximul 8320™ from Stepan Corp.), 2 parts of a tridecyl alcohol phos ester POE 3 (Stepfac 8181™ from Stepan Corp.), 0.2 parts of a polysiloxane defoamer, 1 part of BHT (antioxidant), and about 30 parts of water. The resulting abamectin emulsion concentrate is a stable solution under a variety of environmental conditions and readily forms a microemulsion when admixed with large quantities of water to dilute the active ingredient to concentrations appropriate for agricultural use.

The above examples are intended to illustrate certain aspects and utilities of the invention, but are not intended to limit the invention in any way.

1. A concentrated stable emulsifiable formulation comprising: A) an amide, B) a water-soluble non-ionic emulsifier, C) a water-insoluble anionic emulsifier, and D) a water-insoluble biocidal azole, wherein the concentrated stable emulsifiable formulation is a solution containing the water-insoluble biocidal azole in an amount greater than 20% by weight of the formulation.

2. The concentrated stable emulsifiable formulation of claim 1 wherein the amide comprises a compound of formula 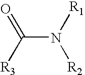
where R₁ and R₂ are independently H, a C₁-C₈ alkyl or alkenyl, a C₁-C₈ alkoxy, a C₁-C₈ alkene, a C₄-C₈ cyclic moiety or C₄-C₁₀ alkaryl, and R₃ is a C₁-C₁₂ alkyl or alkenyl, a C₂-C₁₂ alkoxy, a C₂-C₁₂ alkene, a C₄-C₈ cyclic moiety, a C₆-C₁₂ aryl or alkaryl, a C₆-C₂₀ alkyl ethoxylate or a C₆-C₁₄ fatty acid.

3. The concentrated stable emulsifiable formulation of claim 2 wherein R₁ and R₂ are independently H or a C₁-C₄ alkyl, and R₃ is a C₄-C₁₀ alkyl.

4. The concentrated stable emulsifiable formulation of claim 2 wherein the water-soluble non-ionic emulsifier comprises an alkyl ethoxylate, an alkenyl ethoxylate, an alkylphenol ethoxylate, an alcohol ethoxylate, a fatty acid ethoxylate, an EO/PO block copolymer, or mixture thereof.

5. The concentrated stable emulsifiable formulation of claim 2 wherein the water-soluble non-ionic emulsifier comprises an ethoxylated C₁₂-C₂₄ vegetable oil with between about 20 and about 40 moles of EO per mole of oil.

6. The concentrated stable emulsifiable formulation of claim 2 wherein the water-insoluble anionic emulsifier comprises a metal or alkaline earth metal salt of benzosulfonate substituted in the ortho-, meta-, or para-position with C₈-C₁₆ alkyl or alkenyl, a C₁₀-C₁₈ alkoxy, or a C₈-C₁₆ alkene.

7. The concentrated stable emulsifiable formulation of claim 2 wherein the water-insoluble anionic emulsifier comprises a metal or alkaline earth metal salt of benzosulfonate substituted in the ortho-, meta-, or para-position with C₈-C₁₆ alkyl or alkenyl

8. The concentrated stable emulsifiable formulation of claim 1 containing greater than 25% by weight of the water-insoluble biocidal azole.

9. The concentrated stable emulsifiable formulation of claim 1 wherein the water-insoluble biocidal azole comprises tebuconazole, propiconazole, cyproconazole, tetraconazole, difenoconazole, diniconazole, penconazole, imibenconazole, propiconazole, azaconazole, epoxyconazole, fenbuconazole, fluqinconazole, hexaconazole, imibenconazole, ipconazole, metconazole, penconazole, triticonazole, fluquinconazole, or mixture thereof.

10. The concentrated stable emulsifiable formulation of claim 1 wherein the amide comprises N,N-dialkyloctanamide, N,N-dialkyldecanamide, N-alkyloctanamide, N-alkyldecanamide, octanamide, decanamide, or mixture thereof, where the alkyl groups independently comprise between one and three carbon atoms.

11. The concentrated stable emulsifiable formulation of claim 1 further comprising an N-alkyl-pyrrolidone.

12. The concentrated stable emulsifiable formulation of claim 1 further comprising N-methyl-pyrrolidone, N-heptyl-pyrrolidone, or both.

13. The concentrated stable emulsifiable formulation of claim 2 further comprising an N-alkyl-pyrrolidone.

14. The concentrated stable emulsifiable formulation of claim 1 comprising, per part of the water-insoluble biocidal azole, between 1 and 3 parts of the amide, between 0.08 and 0.4 parts of the water-soluble non-ionic emulsifier, and between 0.03 and 0.3 parts of the water-insoluble anionic emulsifier.

15. The concentrated stable emulsifiable formulation of claim 2 comprising, per part of the water-insoluble biocidal azole, between 1.3 and 2 parts of the amide, between 0.1 and 0.3 parts of the water-soluble non-ionic emulsifier, and between 0.04 and 0.2 parts of the water-insoluble anionic emulsifier.

16. The concentrated stable emulsifiable formulation of claim 1 further comprising a non-azole organic biocide.

17. The concentrated stable emulsifiable formulation of claim 17 wherein the non-azole organic biocide is selected from permethrin, cypermethrin, cyfluthrin, tetramethrin, cyhalothrin, and bifenthrin.

18. The concentrated stable emulsifiable formulation of claim 17 wherein the non-azole organic biocide comprises imidacloprid.

19. The concentrated stable emulsifiable formulation of claim 17 wherein the non-azole organic biocide is selected from fipronil and indoxacarb.

20. The concentrated stable emulsifiable formulation of claim 16 further comprising an N-alkyl-pyrrolidone.

21. An aqueous wood treatment composition comprising emulsified droplets comprising
at least one water-insoluble biocidal azole selected from tebuconazole, propiconazole, cyproconazole, tetraconazole, difenoconazole, diniconazole, penconazole, imibenconazole, propiconazole, azaconazole, epoxyconazole, fenbuconazole, fluqinconazole, hexaconazole, imibenconazole, ipconazole, metconazole, penconazole, triticonazole, and fluquinconazole; and
an amide of formula 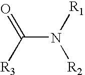
wherein R₁ and R₂ are independently H or a C₁-C₄ alkyl, and R₃ is a C₄-C₁₀ alkyl.

22. The aqueous wood treatment composition of claim 21 wherein the amide is selected from N,N-dialkyloctanamide, N,N-dialkyldecanamide, N-alkyloctanamide, N-alkyldecanamide, octanamide, decanamide, or mixture thereof, where the alkyl groups independently comprise between one and three carbon atoms.

23. The aqueous wood treatment composition of claim 21 further comprising an alkyl ethoxylate, an alkenyl ethoxylate, an alkylphenol ethoxylate, an alcohol ethoxylate, a fatty acid ethoxylate, an EO/PO block copolymer, or mixture thereof.

24. The aqueous wood treatment composition of claim 21 further comprising an ethoxylated C₁₂-C₂₄ vegetable oil with between about 20 and about 40 moles of EO per mole of oil.

25. The aqueous wood treatment composition of claim 21 further comprising a metal or alkaline earth metal salt of benzosulfonate substituted in the ortho-, meta-, or para-position with C₈-C₁₆ alkyl or alkenyl, a C₁₀-C₁₈ alkoxy, or a C₈-C₁₆ alkene.

26. The aqueous wood treatment composition of claim 21 further comprising an N-alkyl-pyrrolidone.

27. The aqueous wood treatment composition of claim 21 further comprising N-methyl-pyrrolidone, N-heptyl-pyrrolidone, or both.

28. The aqueous wood treatment composition of claim 21 further comprising a non-azole organic biocide.

29. The aqueous wood treatment composition of claim 28 wherein the non-azole organic biocide is selected from permethrin, cypermethrin, cyfluthrin, tetramethrin, cyhalothrin, and bifenthrin.

30. The aqueous wood treatment composition of claim 29 further comprising an N-alkyl-pyrrolidone.

31. The aqueous wood treatment composition of claim 28 wherein the non-azole organic biocide is selected from fipronil and indoxacarb.

32. The aqueous wood treatment composition of claim 28 wherein the non-azole organic biocide comprises imidacloprid.

33. The aqueous wood treatment composition of claim 28 wherein the non-azole organic biocide comprises an isothiazolone.

34. The aqueous wood treatment composition of claim 28 wherein the non-azole organic biocide comprises an abamectin.

35. The aqueous wood treatment composition of claim 28 wherein the composition is free of copper.

36. The aqueous wood treatment composition of claim 21 wherein the water-insoluble biocidal azole comprises tebuconazole, further comprising a non-azole organic biocide selected from permethrin, cypermethrin, cyfluthrin, tetramethrin, cyhalothrin, bifenthrin, fipronil, imidacloprid, and indoxacarb.

37. The aqueous wood treatment composition of claim 36 further comprising an N-alkyl-pyrrolidone.

38. The aqueous wood treatment composition of claim 37 wherein the composition is free of copper.

39. A method of preserving wood comprising injecting into said wood an aqueous emulsion comprising
A) at least one water-insoluble biocidal azole selected from tebuconazole, propiconazole, cyproconazole, tetraconazole, difenoconazole, diniconazole, penconazole, imibenconazole, propiconazole, azaconazole, epoxyconazole, fenbuconazole, fluqinconazole, hexaconazole, imibenconazole, ipconazole, metconazole, penconazole, triticonazole, and fluquinconazole
B) an amide of formula 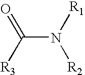
wherein R₁ and R₂ are independently H or a C₁-C₄ alkyl, and R₃ is a C₄-C₁₀ alkyl; and
C) an alkyl ethoxylate, an alkenyl ethoxylate, an alkylphenol ethoxylate, an alcohol ethoxylate, a fatty acid ethoxylate, an EO/PO block copolymer, or mixture thereof.

40. The method of claim 39 wherein the amide is selected from N,N-dialkyloctanamide, N,N-dialkyldecanamide, N-alkyloctanamide, N-alkyldecanamide, octanamide, decanamide, or mixture thereof, where the alkyl groups independently comprise between one and three carbon atoms.

41. The method of claim 39 wherein the aqueous emulsion further comprises a metal or alkaline earth metal salt of benzosulfonate substituted in the ortho-, meta-, or para-position with C₈-C₁₆ alkyl or alkenyl, a C₁₀-C₁₈ alkoxy, or a C₈-C₁₆ alkene.

42. The method of claim 39 wherein the aqueous emulsion further comprises a non-azole organic biocide selected from permethrin, cypermethrin, cyfluthrin, tetramethrin, cyhalothrin, bifenthrin, fipronil, indoxacarb, and imidacloprid.

43. The method of claim 42 wherein the aqueous emulsion further comprises an N-alkyl-pyrrolidone.

44. A method of treating wood comprising
a) admixing the concentrated stable emulsifiable formulation of claim 1 into a concentrated aqueous copper amine solution, said concentrated aqueous copper amine solution having less than about 60% by weight water, wherein the resulting concentrate mixture is a stable solution;
b) adding water to dilute the concentrate mixture to a concentration useful for preserving wood, and providing sufficient shear to create a stable emulsion of the biocidal azole in the diluted copper amine solution; and
c) injecting the stable emulsion of the biocidal azole in the diluted copper amine solution into wood.
